(19) United States
(12) Patent Application Publication
(10) Pub. No.: US 2020/0085777 A1
(43) Pub. Date: Mar. 19, 2020

(54) SOLID PARTICLES

(71) Applicant: DSM IP Assets B.V., Heerlen (NL)

(72) Inventors: Jihane ACHKAR, Kaiseraugst (CH); Bernd SCHLEGEL, Kaiseraugst (CH); Loni SCHWEIKERT, Kaiseraugst (CH); Olivia Brigitte VIDONI, Kaiseraugst (CH)

(21) Appl. No.: 16/613,915

(22) PCT Filed: May 17, 2018
(86) PCT No.: PCT/EP2018/062831

§ 371 Date: Nov. 15, 2019

(30) Foreign Application Priority Data
May 19, 2017 (EP) 17171890.1

(51) Int. Cl. A61K 31/23; A23L 33/155; A23P 10/28; A23L 33/125; A61K 31/222; A61K 9/16

(52) U.S. Cl. CPC A61K 31/23; A23L 33/155; A23P 10/28; A23V 2002/00; A61K 31/222; A61K 9/1623; A23L 33/125

(57) ABSTRACT

The present patent application relates to solid particles comprising vitamin A and/or its derivatives, which are more stable when compressed into tablets.

The present patent application relates to new solid particles comprising a high amount of vitamin A and/or its derivatives (such as vitamin A acetate and vitamin A palmitate) and which do not comprise any antioxidants. The new particles are very stable (against oxidation).

Compressed tablets are a very useful way for administering fat-soluble vitamins. They are easy to be consumed, easy to store and good to handle.

When compressed tablets are produced, harsh conditions are to be applied. It is clear that a certain pressure has to be used to compress any formulation into a tablet. Therefore, there is usually an issue, that the ingredients, which are part of the formulation, which is used to be compressed, are squeezed out and therefore are not part of the tablet anymore. In other words, the tablet contains usually less of the fat-soluble vitamin in the compressed tablet than in the formulation, which was compressed. Usually the content of the fat-soluble vitamins is getting less during the storage of the compressed tablets.

Gelatine, which is often used to formulate fat-soluble vitamins, is usually sourced from an animal source and therefore not suitable for vegetarians.

Furthermore, it is very usual and common to add at least one antioxidant to the solid particle to improve the stability of the particle.

Due to the importance of compressed tablets, comprising vitamin A and/or its derivatives (such as vitamin A acetate and vitamin A palmitate), there is always a need for improved compressible formulations.

Surprisingly it was found that such an improvement was achieved by adding one or more non-reducing sugar and by not adding any antioxidants to the solid formulation, which is used to produce compressed tablets

Therefore, the present invention relates to solid particles (SP) comprising
(i) at least 20 weight-% (wt-%), based on the total weight of the solid particles, of vitamin A and/or its derivatives,
(ii) at least one emulsifier, and
(iii) at least one non-reducing sugar,

wherein the solid particles do not comprise any antioxidants.

Therefore, the present invention relates to solid particles (SP′) comprising
(i) at least 20 weight-% (wt-%), based on the total weight of the solid particles, of vitamin A acetate and/or vitamin A palmitate,
(ii) at least one emulsifier, and
(iii) at least one non-reducing sugar,

wherein the solid particles do not comprise any antioxidants.

These solid particles show better storage stability (of the vitamin A and/or its derivatives (such as vitamin A acetate and vitamin A palmitate)) per se as well when compressed into tablets.

Antioxidants are a class of preservatives, which includes natural antioxidants (such as ascorbic acid, and tocopherols), as well as synthetic antioxidants (such as propyl gallate, tertiary butylhydroquinone, butylated hydroxyanisole and butylated hydroxytoluene).

It is surprising that also without the use of antioxidants the solid particles are equally stable.

It also possible to produce solid particles with only these three kinds of ingredients.

Therefore, the present invention relates to solid particles (SP1) consisting of
(i) at least 22 weight-% (wt-%), based on the total weight of the solid particles, of vitamin A and/or its derivatives,
(ii) at least one emulsifier, and
(iii) at least one non-reducing sugar.

Therefore, the present invention relates to solid particles (SP1′) consisting of
(i) at least 22 weight-% (wt-%), based on the total weight of the solid particles, of vitamin A acetate and vitamin A palmitate,
(ii) at least one emulsifier, and
(iii) at least one non-reducing sugar.

Preferred non-reducing sugars are non-reducing disaccharides; more preferably sucrose and/or trehalose, most preferred is trehalose.

Sucrose is a disaccharide combination of the monosaccharides glucose and fructose with the formula C₁₂H₂₂O₁₁. It is commercially available from many suppliers.

Sucrose is often extracted and refined from either cane or beet sugar for human

Trehalose, also known as mycose or tremalose, is a natural alpha-linked disaccharide formed by an α,α-1,1-glucoside bond between two α-glucose units. There is an industrial process where trehalose is derived from corn starch. There are known biological pathways for trehalose biosynthesis.

Trehalose is available commercially from various suppliers.

The amount of non-reducing sugar in the solid particles is from 5-55 weight-% (wt-%), based on the total weight of the solid particles. Preferably 10-50 wt-%, based on the total weight of the solid particles; more preferably 15-45 wt-%, based on the total weight of the solid particles.

Therefore the present invention relates to solid particles (SP2), which are solid particles (SP), (SP′), (SP1) or (SP1′) comprising 5-55 wt-%, based on the total weight of the solid particles, of at least one non-reducing sugar.

Therefore the present invention relates to solid particles (SP3), which are solid particles (SP), (SP′), (SP1), (SP1′) or (SP2) comprising 10-50 wt-%, based on the total weight of the solid particles, of at least one non-reducing sugar.

Therefore the present invention relates to solid particles (SP4), which are solid particles (SP) or (SP′) comprising 15-45 wt-%, based on the total weight of the solid particles, of at least one non-reducing sugar.

The solid particles according to the present invention comprise usually comprise 22-75 wt-%, based on the total weight of the solid particles, of vitamin A and/or its derivatives (such as vitamin A acetate and vitamin A palmitate), preferably, 25-65 wt-%, based on the total weight of the solid particles.

Therefore, the present invention relates to solid particles (SP5), which are solid particles (SP), (SP′), (SP1), (SP1′), (SP2), (SP3) or (SP4), wherein the solid particles comprise 22-75 wt-%, based on the total weight of the solid particles, of vitamin A and/or its derivatives.

Therefore, the present invention relates to solid particles (SP6), which are solid particles (SP), (SP′), (SP1), (SP1′), (SP2), (SP3), (SP4) or (SP5), wherein the solid particles comprise 25-65 wt-%, based on the total weight of the solid particles, of vitamin A and/or its derivatives.

Furthermore, the solid particles according to the present invention comprise at least one emulsifier. Any commonly known and used emulsifier can be used. A single emulsifier as well as a mixture of emulsifiers can be used.

Suitable emulsifiers are modified (food) starches, ascorbyl palmitate, pectin, alginate, carrageenan, furcellaran, dextrin derivatives, celluloses and cellulose derivatives (e.g. cellulose acetate, methyl cellulose, hydroxypropyl methyl cellulose), lignosulfonate, polysaccharide gums (such as gum acacia (=gum arabic), modified gum acacia, TIC gum, flaxseed gum, ghatti gum, tamarind gum and arabinogalactan), gelatine (bovine, fish, pork, poultry), plant proteins (such as are for example peas, soybeans, castor beans, cotton, potatoes, sweet potatoes, manioc, rapeseed, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, and sugar ester (as well as derivatives thereof).

Preferred are emulsifiers, which are not derived from an animal source.

More preferred emulsifiers are modified (food) starches, polysaccharide gums and plant proteins.

The starches can be modified physically and chemically. Pregelatinized starches are examples of physically modified starches. Acidic modified, oxidized, cross-linked, starch esters, starch ethers and cationic starches are examples of chemically modified starches.

The amount of the emulsifier(s) in the solid particles is usually from 20-70 wt-%, based on the total weight of the solid particles; preferably 25-65 wt-%, based on the total weight of the solid particles.

Therefore the present invention relates to solid particles (SP7), which are solid particles (SP), (SP′), (SP1), (SP1′), (SP2), (SP3), (SP4), (SP5) or (SP6), wherein the at least emulsifier is chosen from the group consisting of modified (food) starches, ascorbyl palmitate, pectin, alginate, carrageenan, furcellaran, dextrin derivatives, celluloses and cellulose derivatives (e.g. cellulose acetate, methyl cellulose, hydroxypropyl methyl cellulose), lignosulfonate, polysaccharide gums (such as gum acacia (=gum arabic), modified gum acacia, TIC gum, flaxseed gum, ghatti gum, tamarind gum and arabinogalactan), gelatine (bovine, fish, pork, poultry), plant proteins (such as are for example peas, soybeans, castor beans, cotton, potatoes, sweet potatoes, manioc, rapeseed, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, and sugar ester (as well as derivatives thereof).

Therefore the present invention relates to solid particles (SP7′), which are solid particles (SP), (SP′), (SP1), (SP1′), (SP2), (SP3), (SP4), (SP5) or (SP6), wherein the at least emulsifier is not derived from an animal source.

Therefore the present invention relates to solid particles (SP7″), which are solid particles (SP), (SP′), (SP1), (SP1′), (SP2), (SP3), (SP4), (SP5) or (SP6), wherein the at least emulsifier is chosen from the group consisting of modified (food) starches, polysaccharide gums and plant proteins.

Therefore the present invention relates to solid particles (SP8), which are solid particles (SP), (SP′), (SP1), (SP1′), (SP2), (SP3), (SP4), (SP5), (SP6), (SP7), (SP7′) or (SP7″), wherein the amount of the emulsifier(s) in the solid particles is 20-70 wt-%, based on the total weight of the solid particles.

Therefore the present invention relates to solid particles (SP9), which are solid particles (SP), (SP′), (SP1), (SP1′), (SP2), (SP3), (SP4), (SP5), (SP6), (SP7), (SP7′) or (SP7″), wherein the amount of the emulsifier(s) in the solid particles is 25-65 wt-%, based on the total weight of the solid particles.

Furthermore, the solid particles can comprise further ingredients (auxiliary agents). It is clear that these auxiliary agents do not include any antioxidants.

Such auxiliary agents are for example gel-forming agents (such as xanthan gum or gellan gum); humectants (such as glycerine, sorbitol, polyethylene glycol); dyes; fragrances; fillers and buffers.

These auxiliary agents can be useful for the solid particles, for their production, for the final product (for what the solid particles used) and/or for the production of the final product.

These compounds can optionally be used in an amount of up to 15 wt-%, based on the solid particles.

Therefore the present invention relates to solid particles (SP10), which are solid particles (SP), (SP′), (SP1), (SP1′), (SP2), (SP3), (SP4), (SP5), (SP6), (SP7), (SP7′), (SP7″), (SP8) or (SP9), comprising up to 15 wt-%, based on the solid particles, of at least one auxiliary agents.

Therefore the present invention relates to solid particles (SP11), which are solid particles (SP10), wherein the auxiliary agent (or auxiliary agents) is chosen from the group consisting of gel-forming agents (such as xanthan gum, gellan gum); humectants (such as glycerine, sorbitol, polyethylene glycol); dyes; fragrances; fillers and buffers.

Depending on the way of the production of the solid particles according to the present invention it also possible that they are coated with a powder, which is used in the powder catch process. Such a powder can be for example corn starch.

The amount of the powder (especially of corn starch) can be up to 15 wt-%, based on the total weight of the powder coated particles. Usually the content of the powder coating is kept as low as possible, so that another coating layer can be created.

Furthermore, it is also possible to coat the solid particles with a coating layer. This layer can be of any known and used coating material.

A suitable size of the solid particles of the present invention is between 50-1000 μm (preferably 100-800 μm); the size is defined by the diameter of the longest dimension of the particle and measured by commonly known method (like laser diffraction)

All particle sizes of the solid particles according to the present invention are determined by laser diffraction technique using a “Mastersizer 3000” of Malvern Instruments Ltd., UK. Further information on this particle size characterization method can e.g. be found in “Basic principles of particle size analytics”, Dr. Alan Rawle, Malvern Instruments Limited, Enigma Business Part, Grovewood Road, Malvern, Worcestershire, WR14 1XZ, UK and the “Manual of Malvern particle size analyzer”. Particular reference is made to the user manual number MAN 0096, Issue 1.0, November 1994. If nothing else is stated all particle sizes referring to the coarse particles of the solid particles according to the present invention are Dv90 values (volume diameter, 90% of the population resides below this point, and 10% resides above this point) determined by laser diffraction. The particle size can be determined in the dry form, i.e. as powder or in suspension. Preferably, the particle size of the solid particles according to the present invention is determined as powder.

The distribution of the particle size of the solid particles is also no essential feature of the present invention.

The shape of the solid particles is also not an essential feature of the present invention. The shape can be sphere-like or any other form (also mixtures of shapes). Usually and preferably, the particles are sphere-like.

The particles can be produced by any commonly known process, which are used to produce such particles (spray drying, spray chilling, etc.).

The process of coating such small particles is well known. It is usually done by fluidized bed spray granulation, film coating or wet granulation.

The solid particles according to the present invention are mainly used for producing compressed tablet.

Therefore the present invention relates to the use of at least one solid particle (SP), (SP′), (SP1), (SP1′), (SP2), (SP3), (SP4), (SP5), (SP6), (SP7), (SP7′), (SP7″), (SP8), (SP9), (SP10) and/or (SP11) in the production of compressed tablets.

The pressure, which is used to producing tablets, is at least 5 kN

The pressure, which is used to producing tablets, is usually between 5 and 40 kN, preferably between 10-40 kN, more preferably between 5-40 kN.

Therefore the present invention relates to the process (P) of producing compressed tables wherein at least one solid particle (SP), (SP′), (SP1), (SP1′), (SP2), (SP3), (SP4), (SP5), (SP6), (SP7), (SP7′), (SP7″), (SP8), (SP9), (SP10) and/or (SP11) are compressed with at pressure of at least 5 kN.

Therefore the present invention relates to the process (P′) of producing compressed tables wherein at least one solid particle (SP), (SP′), (SP1), (SP1′), (SP2), (SP3), (SP4), (SP5), (SP6), (SP7), (SP7′), (SP7″), (SP8), (SP9), (SP10) and/or (SP11) are compressed with at pressure of between 5 and 40 kN,

Therefore the present invention relates to the process (P″) of producing compressed tables wherein at least one solid particle (SP), (SP′), (SP1), (SP1′), (SP2), (SP3), (SP4), (SP5), (SP6), (SP7), (SP7′), (SP7″), (SP8), (SP9), (SP10) and/or (SP11) are compressed with at pressure of between 10-40 kN.

Therefore the present invention relates to the process (P′″) of producing compressed tables wherein at least one solid particle ((SP), (SP′), (SP1), (SP1′), (SP2), (SP3), (SP4), (SP5), (SP6), (SP7), (SP7′), (SP7″), (SP8), (SP9), (SP10) and/or (SP11) are compressed with at pressure of between 15-40 kN.

It is also possible to add any further ingredients (such as fillers, dyestuffs, antioxidants, flavours, etc.) to the solid particles according to the present invention before compressing the particles into the tablet.

Therefore the present invention relates to the process (P1), which is process (P), (P′), (P″) or (P′″), wherein at least one further ingredient is added.

The tablet can be a dietary supplement or a pharmaceutical product. This depends what is added to the compressed tablets additionally.

Furthermore the present invention also relates to compressed tablets comprising at least one solid particle (SP), (SP′), (SP1), (SP1′), (SP2), (SP3), (SP4), (SP5), (SP6), (SP7), (SP7′), (SP7″), (SP8), (SP9), (SP10) and/or (SP11).

The invention is illustrated by the following Example. All temperatures are given in ° C. and all parts and percentages are related to the weight.

### EXAMPLES

### Example 1

370.6 g of deionized water were heated up to 60° C.-65° C. in a vessel. 324.00 g of food modified starch and 121.2 g of trehalose were added and the mixture was brought into solution while stirring at 60-65° C. The obtained solution was cooled to 50-55° C. and degassed for 1 hour. Thereupon, 188.78 g vitamin A acetate) were added to the matrix system and emulsified. The temperature of the process was always kept below 65° C. After emulsification the inner phase of the emulsion had an average particle size of about 272 nm (Dv(0.1)=100 nm, Dv(0.5)=272 nm, Dv(0.9)=559 nm), measurement realized by laser diffraction (Malvern 3000). After emulsification the moisture of the emulsion, determined by a halogen moisture analyzer (Mettler Toledo, Type HR73-P), was checked and adapted if necessary. Afterwards 150 g of the emulsion were sprayed into a spray pan containing 1500 g of corn starch using a rotating spray nozzle. The obtained particles were sieved off (150 to 600 μm) from the excess of corn starch and dried at room temperature using a stream off air. The final product particle size after drying was in average 246 μm (Dv(0.1)=198 μm, Dv(0.5)=246 μm, Dv(0.9)=303 μm) measured by laser diffraction (Malvern 3000).

Solid particles with the composition as listed in the table 1 have been obtained.

TABLE 1
The solid particle according to the present invention

| Ingredient | Amount [wt %] |
|---|---|
| Vitamin A Acetate (2.8 Mio I.U/g) | 27.00 |
| Food modified starch | 48.39 |
| Trehalose | 18.61 |
| Corn Starch | 4.00 |
| Water | 2.00 |

### Example 2: Composition with an Antioxidant (Comparison Example)

The same procedure as described hereinbefore was followed using 190.82 g of an oil mixture (188.78 g vitamin A acetate, 1.04 g BHT) and using less modified food starch (316.75 g))

TABLE 2
The solid particle comprising an antioxidant (BHT)

| Ingredient | Amount [wt %] |
|---|---|
| Vitamin A Acetate (2.8 Mio I.U/g) | 27.00 |
| BHT | 1.5 |
| Food modified starch | 47.31 |
| Trehalose | 18.19 |
| Corn Starch | 4.00 |
| Water | 2.00 |

### Example 3

This example was done in analogy to example 1, but sucrose was used instead of trehalose.

### Example 4 (Comparison Example)

This example was done in analogy to example 2, but sucrose was used instead of trehalose.

All solid particles (from Example 1, 2 3 and 4) were tested. The results are summarised in the following tables.

TABLE 3
Per se stability of the solid particles (stored for 12 months)

| Solid | Content of Vitamin A acetate | Content of Vitamin A |
|---|---|---|
| particles | stored at 25° C. [%] | acetate stored at 30° C. [%] |
| Exp. 1 | 90 | 82 |
| Exp. 2 | 90 | 83 |
| Exp. 3 | 90 | 81 |
| Exp. 4 | 84 | 81 |

### Example 5: Stability in Stress Tablets

100 g of powder consisting of 27 g of vitamin A acetate particles (as obtained in Example 1 and 2), 33.24 g microcrystalline cellulose, 49.86 g calcium phosphate and 0.2 g of magnesium stearate was mixed during 10 min. This end preparation was then compressed with a pressure of 35 KN. The tablets (common disk-shaped; 0.2 g) were stored at room temperature in a closed brown-glass bottle and the vitamin A acetate content determined after 24 months of storage.

It was found that after that period of storage time, surprisingly the amount of Vitamin A acetate was for both tablets at 64%. It could be seen that the solid particles without antioxidant are as stable as the ones with an antioxidant.

1. Solid particles comprising
(i) at least 20 wt-%, based on the total weight of the solid particles, of vitamin A and/or its derivatives,
(ii) at least one emulsifier, and
(iii) at least one non-reducing sugar,
wherein the solid particles do not comprise any antioxidants.

2. Solid particles according to claim 1 comprising
5-55 weight-% (wt-%), based on the total weight of the solid particle, of at least one non-reducing sugar (preferably trehalose).

3. Solid particles according to claim 1 comprising
10-50 weight-% (wt-%), based on the total weight of the solid particle, of at least one non-reducing sugar.

4. Solid particles according to claim 1, wherein the vitamin a derivative is chosen from the group consisting of vitamin A acetate or vitamin A palmitate.

5. Solid particles according to claim 1 comprising 22-75 wt-%, based on the total weight of the solid particle, of vitamin A and/or its derivatives.

6. Solid particles according to claim 1 comprising 25-65 wt-%, based on the total weight of the solid particle, of vitamin A and/or its derivatives.

7. Solid particles according to claim 1 comprising 20-70 wt-%, based on the total weight of the solid particles, of at least one emulsifier.

8. Solid particles according to claim 1, wherein the at least emulsifier is chosen from the group consisting of modified (food) starches, ascorbyl palmitate, pectin, alginate, carrageenan, furcellaran, dextrin derivatives, celluloses and cellulose derivatives (e.g. cellulose acetate, methyl cellulose, hydroxypropyl methyl cellulose), lignosulfonate, polysaccharide gums (such as gum acacia (=gum arabic), modified gum acacia, TIC gum, flaxseed gum, ghatti gum, tamarind gum and arabinogalactan), gelatine (bovine, fish, pork, poultry), plant proteins (such as are for example peas, soybeans, castor beans, cotton, potatoes, sweet potatoes, manioc, rapeseed, sunflowers, sesame, linseed, safflower, lentils, nuts, wheat, rice, maize, barley, rye, oats, lupin and sorghum), animal proteins including milk or whey proteins, lecithin, polyglycerol ester of fatty acids, monoglycerides of fatty acids, diglycerides of fatty acids, sorbitan ester, and sugar ester (as well as derivatives thereof).

9. Use of the solid particles according claim 1 in the production of compressed tablets.

10. Compressed tablets comprising at least one solid particle according to claim 1.
